(19) United States
(12) Patent Application Publication
(10) Pub. No.: US 2017/0189660 A1
(43) Pub. Date: Jul. 6, 2017

(54) MICRONEEDLE FOR INHIBITING DEFORMATION AND DEGENERATION IN MOISTURE ENVIRONMENT AND MANUFACTURING METHOD THEREOF

(71) Applicant: Sun Young BAEK, Seoul (KR)

(72) Inventor: Sun Young BAEK, Seoul (KR)

(21) Appl. No.: 15/158,820

(22) Filed: May 19, 2016

(30) Foreign Application Priority Data
Dec. 30, 2015 (KR) 10-2015-0190281

(51) Int. Cl. A61M 37/00; A61K 47/12; B29C 39/02; B05D 1/00; B29C 39/36; B29C 39/26; B29C 39/38; A61K 9/00; B29C 39/42

(52) U.S. Cl. CPC A61M 37/0015; A61K 9/0021; A61K 47/12; B29C 39/028; B29C 39/42; B29C 39/36; B29C 39/26; B29C 39/38; B05D 1/00; A61M 2037/0053; A61M 2037/0023; B29K 2995/006; B29L 2031/7544

(57) ABSTRACT

Disclosed is a microneedle, the microneedle including a needle body portion formed using a water-soluble material; an active ingredient coating portion configured to coat the surface of the needle body portion, and including an active ingredient transferred to the biological tissue; and a waterproof coating portion configured to coat at least a portion of the surface of the active ingredient coating portion, and to protect the needle body portion and the active ingredient coating portion from moisture.

### CROSS-REFERENCE TO RELATED APPLICATION

This application claims the priority benefit of Korean Patent Application No. 10-2015-0190281, filed on Dec. 30, 2015, in the Korean Intellectual Property Office, the disclosure of which is incorporated herein by reference.

### BACKGROUND

1. Field of the Invention

Example embodiments relate to a microneedle system for inhibiting deformation and degeneration in a moisture environment and a method of manufacturing a microneedle. More particularly, example embodiments relate to a microneedle system for protecting and maintaining a shape of a medical substance from a moisture environment by coating the surface of a melting-type microneedle or a coating microneedle with a coating solution and a method of manufacturing a microneedle.

2. Related Art

A transdermal drug composition method according to the related art has generally employed a method of penetrating the skin within a mouth using a syringe. This method may cause pain and significant stress on a patient side.

In the recent times, research on a microneedle has been actively conducted as a new transdermal delivery method to outperform the above issue. In general, if the microneedle is a device for supplying the nutrition or facilitating the penetration of a medical substance by stimulating the skin or making a small hole on the skin using a small size of a needle, a type of the microneedle is not particularly limited. In general, the microneedle has a diameter of about 1 μm to about 100 μm.

When using the microneedle configured as above, the medical substance may be locally delivered to the cell or tissue without causing relatively significant pain. Further, the microneedle may be effectively used to administer molecules, such as peptide, protein, oligonucleotide, DNA, etc., incapable of infiltrating into the biological membrane or to target-deliver the medical substance to a part hard to be treated and a specific portion or tissue of a body.

The microneedle may have four types, for example, a solid microneedle, a coating microneedle, a melting-type microneedle, a hollow microneedle.

Here, the melting-type microneedle employs a method in which, once the melting-type microneedle is applied to the skin, a microneedle material starts being melted and a medical substance contained therein is delivered to the skin. Using this microneedle, the medical substance may be delivered with a one-time administration and the microneedle does not remain and thus, the risk of pollution is absent.

Here, a relatively long period of time, for example, 10 minutes or more, is required to melt the melting-type microneedle due to the moisture in the skin after administration. Since an active ingredient contained in the microneedle is subject to the complete melting of the microneedle, a fixed amount of medical substance may not be readily delivered to the skin. In addition, the melting-type microneedle is to be manufactured based on molding. Thus, a relatively long manufacturing time is required and the active ingredient may vary due to a manufacturing temperature.

Meanwhile, the coating microneedle may be simply manufactured and may quickly deliver a medical substance since the surface of the coating microneedle is coated with the medical substance. However, the needle remains, which may cause pollution.

In particular, in the case of the melting-type microneedle or the coating microneedle according to the related art, the microneedle is manufactured based on a water-soluble property. Thus, when the microneedle is exposed in an environment in which moisture is relatively abundant, a shape of the microneedle may be deformed or a tip of the microneedle may not be maintained in a sharp shape and easily melted, thereby disabling the microneedle.

### SUMMARY

Example embodiments provide a microneedle system that may coat the surface of a water-soluble microneedle containing a medical substance with a material of which a melting point is higher than a room temperature and in which an active ingredient is added and may deliver a fixed amount of active ingredients quickly when the microneedle is administered to the biological tissue, and a method of manufacturing a microneedle.

Example embodiments also provide a microneedle system that may coat the surface of a water-soluble microneedle with a biodegradable material, such as biodegradable fat, safe to a living body so that the water-soluble microneedle may not easily lose a property as the microneedle due to a tip of the microneedle becoming blunt and the microneedle may readily deliver a medical substance to the inside of the tissue by piercing into the surface of the skin, regardless of being exposed in a moisture environment, and a method of manufacturing a microneedle.

Example embodiments also provide a microneedle system that may use a biocompatible material capable of being melted in the tissue for a microneedle material and coating materials, and may coat the surface of a melting-type microneedle or a coating microneedle with a biodegradable and hydrophobic material, thereby protecting the same in a moisture environment, and a method of manufacturing a microneedle.

Example embodiments also provide a microneedle system that may deliver a fixed amount of active ingredients within a relatively short period of time within a minimum number of administrations, and a method of manufacturing a microneedle.

According to an aspect, there is provided a microneedle including a needle body portion formed using a water-soluble material, an active ingredient coating portion configured to coat the surface of the needle body portion, and including an active ingredient transferred to the biological tissue, and a waterproof coating portion configured to coat at least a portion of the surface of the active ingredient coating portion, and to protect the needle body portion and the active ingredient coating portion from moisture.

Also, the needle body portion may include at least one of a viscous material and the active ingredient transferred to the biological tissue.

Also, the viscous material included in the needle body portion may include at least one of carboxymethyl cellulose(CMC), hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polylactic acid (PLA), polyglycolide (PGA), poly lactic-co-glycolic acid (PLGA), pullulan polyanhydride, polyorthoester, polyetherester, polycarprolactone, polyesteramide, poly (butyric acid), poly (valeric acid), polyurethane, polyacrylate, ethylene-vinyl acetate polymer, acrylic substituted cellulose acetate, non-degradable polyurethane, polystyrene, polyvinyl chloride, polyvinylidene fluoride, poly (vinyl imidazole), chlorosulphonate polyolefins, polyethylene oxide, poly vinyl pyrrolidone (PVP), polyethylene glycol (PEG), polymethacrylate, hydroxypropyl methyl cellulose (HPMC), ethylcellulose (EC), hydroxypropyl cellulose (HPC), carboxymethyl cellulose, cyclodextrin, copolymer of monomers constituting the polymer, and cellulose.

Also, the needle body portion may be formed using biodegradable polymer resin. Also, the active ingredient coating portion may include a viscous material having a melting point greater than or equal to a room temperature and the active ingredient transferred to the biological tissue.

Also, the viscous material included in the active ingredient coating portion may include at least one of maltose, lactose, trehalose, cellobiose, isomaltose, turanose, and lactulose.

Also, the viscous material may be included in the needle body portion, and a solubility of the viscous material included in the active ingredient coating portion against the moisture may be greater than a solubility of the viscous material included in the needle body portion against the moisture.

Also, the active ingredient included in the needle body portion and the active ingredient coating portion may include at least one of α-interferon, β-interferon for multiple sclerosis (MS), erythropoietin, follitropin β, follitropin α, G-CSF, GM-CSF, human chorionic gonadotropin, luteinizing hormone, salmon calcitonin, glucagon, GNRH antagonist, insulin, human growth hormone, filgrastim, heparin, low-molecular-weight heparin, somatropin, Japanense encephalitis vaccine, rotavirus vaccine, Alzheimer vaccine, artery hardening vaccine, cancer vaccine, nicotine vaccine, diphtheria vaccine, tetanus vaccine, pertussis vaccine, lyme disease vaccine, rabies vaccine, dipolcoccus pneumoniae vaccine, yellow fever vaccine, cholera vaccine, vaccinations-exanthem vaccine, tuberculosis (TB) vaccine, German measles vaccine, measles vaccine, epidemic parotitis vaccine, botulinus vaccine, herpesvirus vaccine, other DNA vaccine, hepatitis (type) B vaccine, hyaluronic acid, coenzymeq10, chitosan, botox, vitamin and vitamin derivatives, hydroxy acid, tetracycline, oxytetracycline, doxycycline, minocycline, benzocaine, mepivacaine, lidocaine, prilocaine, bupivacaine, etidocaine, articaine, procaine, propoxycaine, tetracaine, ropivacaine, butacaine, piperocaine, cocaine, chloroprocaine, proparacaine, and dyclonine.

Also, the waterproof coating portion may include a lipid-based material or a mineral-based material.

Also, the lipid-based material or mineral-based material included in the waterproof coating portion may include at least one of beeswax, oleic acid, soy fatty acid, castor oil, phosphatidylcholine, d-α-tocopherol/vitamin E, corn oil mono-di-tridiglycerides, castor oil, corn oil, cottonseed oil, olive oil, peanut oil, peppermint oil, safflower oil, sesame oil, soybean oil, hydrogenated vegetable oil, hydrogenated soybean oil, caprylic/capric triglycerides derived from coconut oil or palm see oil, and phosphatidylcholine.

According to another aspect, there is provided a method of manufacturing a microneedle, the method including manufacturing a needle body portion of the microneedle to be in a preset shape, preparing a coating solution in which a viscous material having a melting point greater than or equal to a room temperature and an active ingredient transferred to the biological tissue are mixed, coating the needle body portion of the micro needle with the coating solution, forming an active ingredient coating portion on the surface of the needle body portion of the microneedle by drying the needle body portion of the microneedle coated with the coating solution at the room temperature, coating at least a portion of the surface of the active ingredient coating portion with a waterproof coating portion, and drying the waterproof coating portion at the room temperature.

Also, the manufacturing of the needle body portion of the microneedle may include preparing a mold for molding the needle body portion of the microneedle, molding the needle body portion of the microneedle, and separating the needle body portion of the microneedle from the mold.

Also, the molding of the needle body portion of the microneedle may include manufacturing the needle body portion of the microneedle by filling the mold with the viscous material and the active material transferred to the biological tissue and drying the same, or by applying a centrifugal process.

Also, the molding of the needle body portion of the microneedle may include filling the mold with the viscous material and the active ingredient transferred to the biological tissue and manufacturing the needle body portion of the microneedle using a vacuum pump. Also, the molding of the needle body portion of the microneedle may include heating biodegradable polymer resin at a preset temperature in a vacuum oven.

Also, the mold may be a structure that includes at least one of polydimethylsiloxane (PDMS), a type of polymer used for the mold, polyurethane, metal, an aluminum biocompatible material, water-soluble polymer, fat-soluble polymer, and amphiphilic polymer. The fat-soluble polymer and the amphiphilic polymer may include at least one of hydroxypropyl cellulose (HPC), hydroxypropyl methyl cellulose (HPMC), polycaprolactone (PCL), polyglycolide (PGA), polylactic acid (PLA), poly lactic-co-glycolic acid (PLGA) polymer, poly vinyl pyrrolidone (PVP), polyethylene glycol (PEG), poly ethylene oxide (PEO), poly propylene oxide (PPO), poly vinyl methyl ether (PVME), PMA (poly (methyl) acrylate)s, propylene glycol, poly (ester amide), poly (butyric acid), acrylamide (acrylic amide), acrylic acid, hyaluronic acid (HA), and gelatin.

Also, the preparing of the coating solution may include mixing the viscous material and the active ingredient at a preset ratio, and melting the mixture of the viscous material and the active ingredient at a temperature greater than or equal to the melting point of the viscous material.

The coating of the needle body portion of the microneedle with the coating solution may include dip-coating or entirely coating the needle body portion of the microneedle with the coating solution.

Also, the coating solution may be a lipid-based or mineral-based coating solution.

Also, the drying of the waterproof coating portion at the room temperature may include coating the surface of the active ingredient coating portion with a lipid-based material or a mineral-based material using an ultrasonic spray coating scheme, an automization coating scheme, or an electro-spinning coating scheme, or dip-coating the needle body portion of the microneedle with a lipid-based or mineral-based coating solution.

According to example embodiments, since a microneedle is coated with a lipid-based material or a mineral-based material, a shape of a tip of the microneedle may be maintained regardless of contact with the moisture and the microneedle may be used even in a moisture environment.

Also, the microneedle according to example embodiments may deliver a fixed amount of active ingredients within a relatively short period of time, for example, a few seconds or a few minutes with a one-time administration.

Also, since a frame of the microneedle is manufactured using a water-soluble material, the microneedle may be partially or completely melted after being administered to the skin tissue. Accordingly, the risk of pollution may be removed.

Also, according to example embodiments, the water-soluble microneedle may be manufactured easily and the economical mass production thereof is enabled.

Also, since a variety of water-soluble viscous materials having a melting point is used as a material used to coat the microneedle, a relatively long period of time is not required until the moisture evaporates and a relatively fast dry is enabled at a room temperature.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a cross-sectional view of a microneedle system according to example embodiments;

FIG. 2 is a flowchart illustrating a method of manufacturing a microneedle according to example embodiments;

FIG. 3 is a flowchart illustrating an operation of manufacturing a needle body portion of a microneedle according to example embodiments;

FIG. 4 is a perspective view illustrating a mold for manufacturing a microneedle according to example embodiments;

FIG. 5 is a perspective view illustrating a state in which a raw material for manufacturing the microneedle or biodegradable polymer resin is injected in the mold of FIG. 4;

FIG. 6 is a perspective view illustrating a needle body portion and a base portion of the microneedle manufactured by removing the mold of FIG. 4;

FIG. 7 is a flowchart illustrating an operation of preparing a coating solution containing an active ingredient of a microneedle according to example embodiments;

FIG. 8 illustrates an example of mixing a viscous material having a melting point greater than or equal to a room temperature and an active ingredient at a preset ratio and melting the mixture at a temperature of the melting point or more to prepare a coating solution of an active ingredient coating portion of a microneedle according to example embodiments;

FIG. 9 illustrates a state in which a needle body portion of a microneedle is dipped into a coating solution containing an active ingredient according to example embodiments;

FIG. 10 illustrates an example of drying a microneedle coated with an active ingredient coating portion at a room temperature according to example embodiments;

FIG. 11 illustrates an example of a process of coating a dried microneedle with a lipid-based material or a mineral-based material using an ultrasonic spray coating scheme or an electro-spinning coating scheme according to example embodiments;

FIG. 12 illustrates an example of dipping a dried microneedle into a lipid-based material or a mineral-based material according to example embodiments;

FIG. 13 illustrates an example of drying a microneedle coated with a lipid-based material or a mineral-based material at a room temperature according to example embodiments;

FIGS. 14 and 15 illustrate an example of comparing a shape of a general melting-type microneedle and a shape of a microneedle coated with a moisture protection solution in a moist environment after 24 hours; and

FIG. 16 illustrates an example of a microneedle observed after 24 hours by putting a moisture-sensitive microneedle in an oven of 70° C. and performing waterproof coating.

### DETAILED DESCRIPTION OF THE INVENTION

Hereinafter, example embodiments will be described with reference to the accompanying drawings. Herein, thicknesses of lines, sizes of constituent elements, etc., illustrated in the drawings, may be exaggerated for clarity and convenience of description. Further, terms described in the following are ones defined based on functions in the present disclosure and thus, may vary based on the intent of a user or an operator, or custom. Accordingly, the definition of such terms should be made based on the overall description disclosed in the present specification.

Although the terms “first,” “second,” “third,” etc., may be used herein to describe various elements, components, regions, layers, and/or sections, these elements, components, regions, layers, and/or sections, should not be limited by these terms. These terms are only used to distinguish one element, component, region, layer, or section, from another region, layer, or section. Thus, a first element, component, region, layer, or section, discussed below may be termed a second element, component, region, layer, or section, without departing from the scope of this disclosure.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

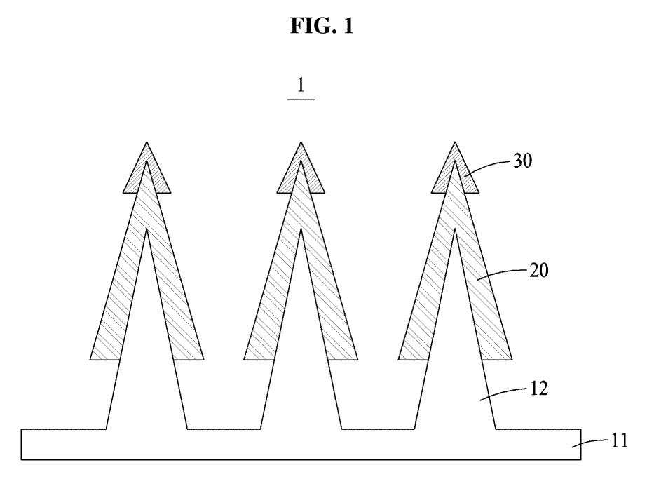

FIG. 1 is a cross-sectional view of a microneedle system according to example embodiments.

Referring to FIG. 1, a microneedle 1 according to example embodiments may include a base portion 11, a needle body portion 12, an active ingredient coating portion 20, and a waterproof coating portion 30.

The base portion 11 may form a bottom surface of the microneedle 1 and may support the needle body portion 12. The base portion 11 may be integrally coupled to the needle body portion 12, thereby configuring a single body. The base portion 11 may be formed using the same material as that of the needle body portion 12.

The base portion 11 may be provided in a thin rectangular shape. A plurality of needle body portions 12 may be disposed on the base portion 11.

An end of the needle body portion 12 may be provided in a sharp conic or quadrangular pyramid shape. A shape of the needle body portion 12 is not particularly limited thereto if the shape allows the penetration of a medical substance into the skin. The needle body portion 12 may be formed in a conic shape with the diameter of about 1 μm to about 100 μm.

The needle body portion 12 may be formed using a water-soluble material. In detail, when the microneedle 1 is applied to a melting-type microneedle, the needle body portion 12 may be formed using the mixture of a viscous material, such as carboxymethyl cellulose (CMC), having viscosity and an active ingredient. In more detail, the needle body portion 12 may be prepared using the viscous material, such as carboxymethyl cellulose (CMC) and the like. The active ingredient is added to the prepared needle body portion 12. As a result, the needle body portion 12 itself may contain the active ingredient. Since the viscous material is included in the needle body portion 12, the mechanical strength of the needle body portion 12 may be enhanced.

As another example, when the microneedle 1 is applied to a coating microneedle, the needle body portion 12 may be formed using biodegradable polymer resin, such as poly lactic acid (PLA) or poly lactic-co-glycolic acid (PLGA).

The viscous material included in the needle body portion 12 may include at least one of carboxymethyl cellulose (CMC), hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polylactic acid (PLA), polyglycolide (PGA), poly lactic-co-glycolic acid (PLGA), pullulan polyanhydride, polyorthoester, polyetherester, polycarprolactone, polyesteramide, poly (butyric acid), poly (valeric acid), polyurethane, polyacrylate, ethylene-vinyl acetate polymer, acrylic substituted cellulose acetate, non-degradable polyurethane, polystyrene, polyvinyl chloride, polyvinylidene fluoride, poly (vinyl imidazole), chlorosulphonate polyolefins, polyethylene oxide, poly vinyl pyrrolidone (PVP), polyethylene glycol (PEG), polymethacrylate, hydroxypropyl methyl cellulose (HPMC), ethylcellulose (EC), hydroxypropyl cellulose (HPC), carboxymethyl cellulose, cyclodextrin, copolymer of monomers constituting the polymer, and cellulose.

The needle body portion 12 may be manufactured by injecting and molding the mixture of the viscous material and the active ingredient into a mold M, or by injecting and molding biodegradable polymer resin into the mold M. A method of manufacturing the needle body portion 12 and a material used for the mold M will be further described below.

The active ingredient coating portion 20 may be used to coat the surface of the needle body portion 12. The active ingredient coating portion 20 may be formed using a material in which the viscous material and the active ingredient are mixed at a preset ratio. A melting point of the viscous material included in the active ingredient coating portion 20 may be greater than or equal to a room temperature, for example, 25° C.

A viscous material having a melting point and a relatively high water-solubility, such as maltose, lactose, trehalose, cellobiose, isomaltose, turanose, lactulose, and the like, may be included as the viscous material included in the active ingredient coating portion 20. Among materials capable of forming the needle body portion 12, carboxymethyl cellulose (CMC), hyaluronic acid, alginic acid, pectin, carrageenan, chondroitin (sulfate), dextran (sulfate), chitosan, polylysine, carboxymethyl chitin, fibrin, agarose, pullulan polylactide, polyanhydride, polyorthoester, polyetherester, polyesteramide, poly (butyric acid), poly (valeric acid), polyacrylate, ethylene-vinyl acetate polymer, acrylic substituted cellulose acetate, polyvinyl chloride, polyvinylidene fluoride, poly (vinyl imidazole), chlorosulphonate polyolefins, polyethylene oxide, poly vinyl pyrrolidone (PVP), hydroxypropyl methyl cellulose (HPMC), ethylcellulose (EC), hydroxypropyl cellulose (HPC), carboxymethyl cellulose, or cyclodextrin may be a viscous material (referred to as a first viscous material) having a relatively low water-solubility. Among materials includable in the active ingredient coating portion 20, maltose, lactose, trehalose, cellobiose, isomaltose, turanose, or lactulose may be a water-soluble material having a relatively high water-solubility, and may b a viscous material (referred to as a second viscous material) having a relatively high water-solubility since they act as the viscous material according to an increase in the concentration thereof.

Also, the active ingredient included in the active ingredient coating portion 20 and the needle body portion 12 may include at least one of α-interferon, β-interferon for multiple sclerosis (MS), erythropoietin, follitropin β, follitropin α, G-CSF, GM-CSF, human chorionic gonadotropin, luteinizing hormone, salmon calcitonin, glucagon, GNRH antagonist, insulin, human growth hormone, filgrastim, heparin, low-molecular-weight heparin, somatropin, Japanense encephalitis vaccine, rotavirus vaccine, Alzheimer vaccine, artery hardening vaccine, cancer vaccine, nicotine vaccine, diphtheria vaccine, tetanus vaccine, pertussis vaccine, lyme disease vaccine, rabies vaccine, dipolcoccus pneumoniae vaccine, yellow fever vaccine, cholera vaccine, vaccinations-exanthem vaccine, tuberculosis (TB) vaccine, German measles vaccine, measles vaccine, epidemic parotitis vaccine, botulinus vaccine, herpesvirus vaccine, other DNA vaccine, hepatitis (type) B vaccine, hyaluronic acid, coenzymeq10, chitosan, botox, vitamin and vitamin derivatives, hydroxy acid, tetracycline, oxytetracycline, doxycycline, minocycline, benzocaine, mepivacaine, lidocaine, prilocaine, bupivacaine, etidocaine, articaine, procaine, propoxycaine, tetracaine, ropivacaine, butacaine, piperocaine, cocaine, chloroprocaine, proparacaine, and dyclonine.

As described above, by distributing the active ingredient, for example, medical substance, over the surface of the needle body portion 12, for example, the active ingredient coating portion 20, a fixed amount of active ingredients, for example, medical substances may be delivered quickly to the inside of the biological tissue. Also, since the active ingredient is included in the active ingredient coating portion 12 and also included in the needle body portion 12 having a volume greater than that of the active ingredient coating portion 12, a large amount of active ingredients may be delivered and the active ingredients may be delivered over a relatively long period of time.

Accordingly, a minimum amount of active ingredients that are to be delivered to the inside of the biological tissue may be included in the active ingredient coating portion 20 and thereby be quickly delivered to the inside of the biological tissue. A remaining amount of active ingredients may be included in the needle body portion 12.

A process of coating and drying the surface of the needle body portion 12 with the active ingredient coating portion 20 will be described below.

A viscous material, such as maltose and the like, included in the active ingredient coating portion 20, has a relatively high water-solubility, that is, a relatively high water-soluble rate, compared to a viscous material, such as CMC and the like, included in the needle body portion 12. Thus, once the microneedle 1 is applied to the biological tissue, the active ingredient included in the active ingredient coating portion 20 may be quickly delivered to the inside of the biological tissue compared to the active ingredient included in the needle body portion 12.

Accordingly, an active ingredient delivery rate to the inside of the biological tissue may be adjusted by adjusting an amount of active ingredients included in the active ingredient coating portion 20 and an amount of active ingredients included in the needle body portion 12.

For example, to accelerate the active ingredient delivery rate, an amount of active ingredients included in the active ingredient coating portion 20 may be adjusted to be greater than an amount of active ingredients included in the needle body portion 12. On the contrary, to relatively decelerate the active ingredient delivery rate, an amount of active ingredients included in the active ingredient coating portion 20 may be adjusted to be less than an amount of active ingredients included in the needle body portion 12.

Also, the active ingredient included in the active ingredient coating portion 20 and the needle body portion 12 may include at least one of α-interferon, β-interferon for multiple sclerosis (MS), follitropin β, follitropin α, G-CSF, GM-CSF, human chorionic gonadotropin, luteinizing hormone, salmon calcitonin, glucagon, GNRH antagonist, insulin, human growth hormone, filgrastim, heparin, low-molecular-weight heparin, somatropin, Japanense encephalitis vaccine, rotavirus vaccine, Alzheimer vaccine, artery hardening vaccine, cancer vaccine, nicotine vaccine, diphtheria vaccine, tetanus vaccine, pertussis vaccine, lyme disease vaccine, rabies vaccine, dipolcoccus pneumoniae vaccine, yellow fever vaccine, cholera vaccine, vaccinations-exanthem vaccine, tuberculosis (TB) vaccine, German measles vaccine, measles vaccine, epidemic parotitis vaccine, botulinus vaccine, herpesvirus vaccine, other DNA vaccine, hepatitis (type) B vaccine, hyaluronic acid, coenzymeq10, chitosan, botox, vitamin and vitamin derivatives, hydroxy acid, tetracycline, oxytetracycline, doxycycline, minocycline, benzocaine, mepivacaine, lidocaine, prilocaine, bupivacaine, etidocaine, articaine, procaine, propoxycaine, tetracaine, ropivacaine, butacaine, piperocaine, cocaine, chloroprocaine, proparacaine, and dyclonine.

In the case of the active ingredient coating portion 20, a weight % ratio of viscous material:active ingredient may be within the range of 9:1 to 1:9, desirably, within the range of 1:1 to 1:2.

The waterproof coating portion 30 may be used to coat an upper end portion or the entire surface of the active ingredient coating portion 20. The waterproof coating portion 30 may prevent the moisture from directly contacting with the water-soluble active ingredient coating portion 20 or needle body portion 12. If the moisture makes a direct contact with the active ingredient coating portion 20 or the needle body portion 12, a shape of an end of the active ingredient coating portion 20 or the needle body portion 12 may be deformed to be blunt. However, since the direct contact of the active ingredient coating portion 20 or the needle body portion 12 with the moisture is prevented by way of the waterproof coating portion 30, the end of the active ingredient coating portion 20 or the needle body portion 12 may be maintained to be in a pointed and sharp shape.

Accordingly, even in an environment in which a relatively large amount of moisture is present, that is, a moisture environment, the microneedle 1 may pierce the surface of the biological tissue and deliver the active ingredient, such as lidocaine and the like, to the inside of the tissue.

The waterproof coating portion 30 may be formed using a lipid-based material or a mineral-based material, in order not to be easily soluble in the water. For example, the waterproof coating portion 30 may include at least one of beeswax, oleic acid, soy fatty acid, castor oil, phosphatidylcholine, d-α-tocopherol/vitamin E, corn oil mono-di-tridiglycerides, castor oil, corn oil, cottonseed oil, olive oil, peanut oil, peppermint oil, safflower oil, sesame oil, soybean oil, hydrogenated vegetable oil, hydrogenated soybean oil, caprylic/capric triglycerides derived from coconut oil or palm see oil, phosphatidylcholine, polyethylene (PE), polypropylene (PP), polytetrafluoroethylene (PTFE), polymethylmethacrylate (PMMA), ethylene vinyl acetate (EVA), polycaprolactone (PCL), polyurethane (PU), polyethylene terephthalate (PET), polyethylene glycol (PEG), polyvinyl alcohol (PVA), poly lactide (PLA), poly lactic-co-glycolic acid (PLGA) polymer, polyglycolide (PGA), wax (paraffin cholesterol), glycerin, chitin, lecithin, animal beef tallow, vegetable stearin, low-grade saturated fatty acid, monosaturated fatty acid, tristearins, fatty acid mineral salt (zinc, calcium, magnesium stearate), and fatty acid zinc salt (stearic acid, palmitic acid, lauric acid), or includes the mixture thereof.

The waterproof coating portion 30 may be coated by dip-coating the needle body portion 12 coated with the active ingredient coating portion 20 into a lipid-based coating solution, or by applying an ultrasonic spray coating scheme, an automization coating scheme, or an electro-spinning coating scheme to the needle body portion 12 coated with the active ingredient coating portion 20. A process of coating the needle body portion 12 with the waterproof coating portion 30 will be described below.

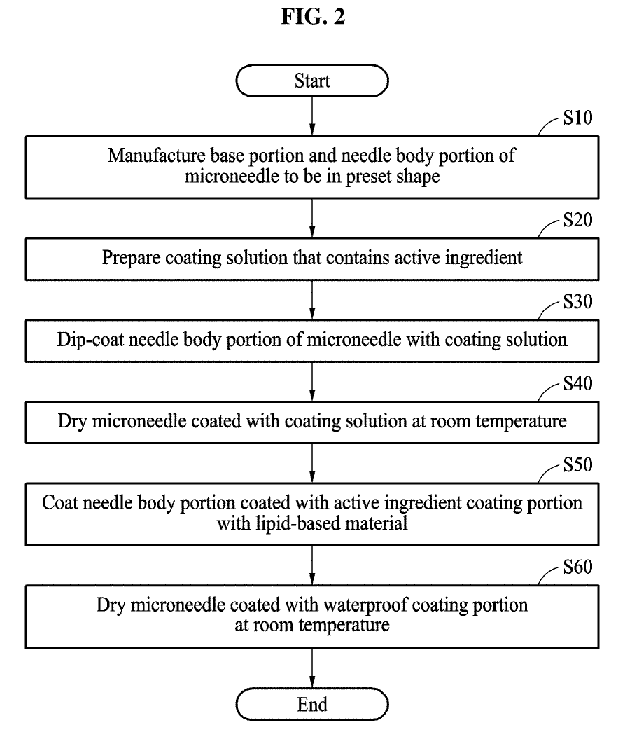

FIG. 2 is a flowchart illustrating a method of manufacturing a microneedle according to example embodiments.

Referring to FIG. 2, to manufacture the microneedle 1 according to example embodiments, the base portion 11 and the needle body portion 12 of the microneedle 1 may be manufactured to be in a preset shape in operation S10. In operation S20, a coating solution of the active ingredient coating portion 20 used to coat the needle body portion 12 may be prepared. In operation S30, the needle body portion 12 may be dip-coated with the coating solution of the active ingredient coating portion 20. In operation S40, the microneedle 1 coated with the coating solution of the active ingredient coating portion 20 may be dried at a room temperature. In operation S50, the needle body portion 12 coated with the active ingredient coating portion 20 may be coated with the waterproof coating portion 30 formed using a lipid-based material or a mineral-based material. In operation S60, the microneedle 1 coated with the waterproof coating portion 30 may be dried at a room temperature.

Hereinafter, each operation will be described with reference to the accompanying drawings.

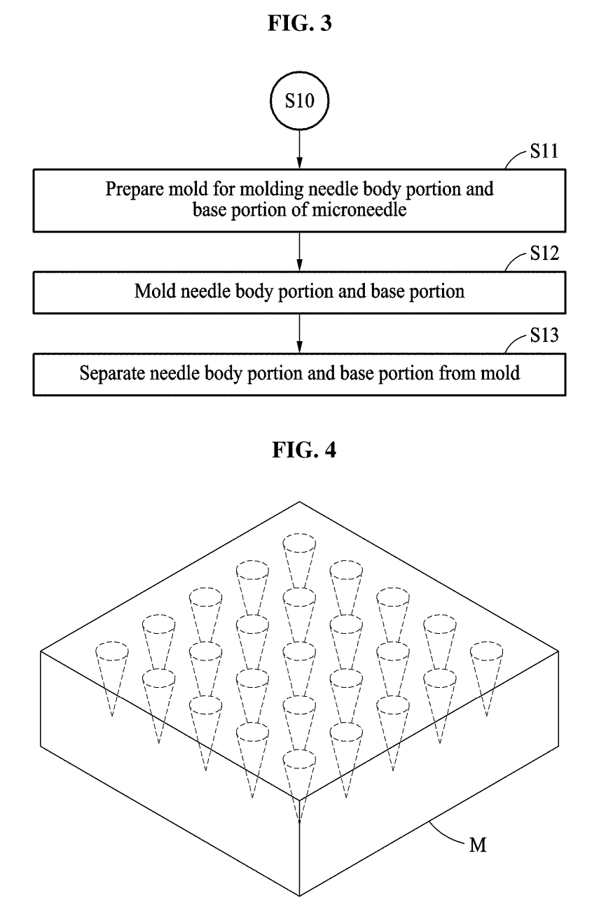

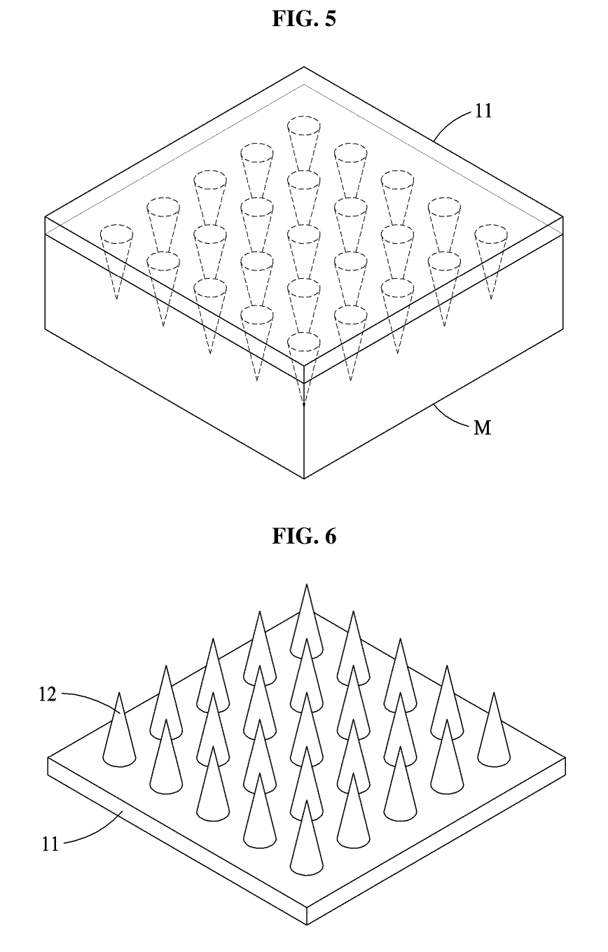

FIG. 3 is a flowchart illustrating an operation of manufacturing a needle body portion of a microneedle according to example embodiments, FIG. 4 is a perspective view illustrating a mold for manufacturing a microneedle according to example embodiments, FIG. 5 is a perspective view illustrating a state in which a raw material for manufacturing the microneedle or biodegradable polymer resin is injected in the mold of FIG. 4, and FIG. 6 is a perspective view illustrating a needle body portion and a base portion of the microneedle manufactured by removing the mold of FIG. 4.

Referring to FIGS. 3 through 6, the base portion 11 and the needle body portion 12 of the microneedle 1 may be manufactured based on molding. Operation S10 of manufacturing the base portion 11 and the needle body portion 12 of the microneedle 1 to be in the present shape may include operation S11 of preparing the mold M for molding the needle body portion 12 and the base portion 11, operation S12 of molding the needle body portion 12 and the base portion 11, and operation S13 of separating the molded needle body portion 12 and the base portion 11 from the mold M.

Operation S10 of manufacturing the base portion 11 and the needle body portion 12 of the microneedle 1 to be in the preset shape may vary depending on whether the microneedle 1 is to be manufactured as a melting-type microneedle or a coating microneedle.

In the case of manufacturing the microneedle 1 as the melting-type microneedle, the needle body portion 12 and the base portion 11 may be manufactured by filling the mold M formed using a material, such as PDMS and the like, with the mixture of a viscous material, such as carboxymethyl cellulose (CMC) and the like, having viscosity, and an active ingredient, and by applying a centrifugal process or a polymer melt process.

As another example, in the case of manufacturing the microneedle 1 as the coating microneedle, the needle body portion 12 and the base portion 11 may be manufactured by filling the mold M with a biodegradable material, such as PLA, PLGA, and the like, and by heating the same in a vacuum oven of about 180° C.

As described above, the needle body portion 12 may include at least one of carboxymethyl cellulose (CMC), hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polylactic acid (PLA), polyglycolide (PGA), poly lactic-co-glycolic acid (PLGA), pullulan polyanhydride, polyorthoester, polyetherester, polycarprolactone, polyesteramide, poly (butyric acid), poly (valeric acid), polyurethane, polyacrylate, ethylene-vinyl acetate polymer, acrylic substituted cellulose acetate, non-degradable polyurethane, polystyrene, polyvinyl chloride, polyvinylidene fluoride, poly (vinyl imidazole), chlorosulphonate polyolefins, polyethylene oxide, poly vinyl pyrrolidone (PVP), polyethylene glycol (PEG), polymethacrylate, hydroxypropyl methyl cellulose (HPMC), ethylcellulose (EC), hydroxypropyl cellulose (HPC), carboxymethyl cellulose, cyclodextrin, and copolymer of monomers constituting the polymer, and cellulose. A plurality of grooves in a conic shape or a quadrangular pyramid shape may be formed on the mold M to manufacture ends of the needle body portions 12 in a sharp shape.

Here, the mold M may be a structure that includes at least one of polydimethylsiloxane (PDMS), a type of polymer used for the mold, polyurethane, metal, an aluminum biocompatible material, water-soluble polymer, fat-soluble polymer, and amphiphilic polymer. The fat-soluble polymer and the amphiphilic polymer may include at least one of hydroxypropyl cellulose (HPC), hydroxypropyl methyl cellulose (HPMC), polycaprolactone (PCL), polyglycolide (PGA), polylactic acid (PLA), poly lactic-co-glycolic acid (PLGA), poly vinyl pyrrolidone (PVP), polyethylene glycol (PEG), poly ethylene oxide (PEO), poly propylene oxide (PPO), poly vinyl methyl ether (PVME), PMA (poly (methyl) acrylate)s, propylene glycol, poly (ester amide), poly (butyric acid), acrylamide (acrylic amide), acrylic acid, hyaluronic acid (HA), and gelatin.

In operation S12 of molding the needle body portion 12 of the microneedle 1, inside bubbles may be removed by filling the PDMS mold M with a solution in which a viscous material, such as carboxymethyl cellulose (CMC) and the like, and an active ingredient transferred to the biological skin tissue are melted and by performing vacuum processing.

Accordingly, in the case of the needle body portion 12, a ratio of viscous material:active ingredient may be within the range of 10:0 to 1:9, desirably, within the range of 2:1 to 3:1.

The needle body portion 12 of the microneedle 1 may be manufactured by remaining only a solution received in each of conic grooves formed on the mold M, by removing a solution remaining on the surface of the mold M, and by naturally drying the solution received in each of the conic or rectangular pyramid grooves formed on the mold M.

Accordingly, when the natural drying is performed with remaining the solution received in the conic grooves formed on the mold M, the needle body portion 12 may be formed on each of the plurality of grooves on the mold M as illustrated in FIG. 6.

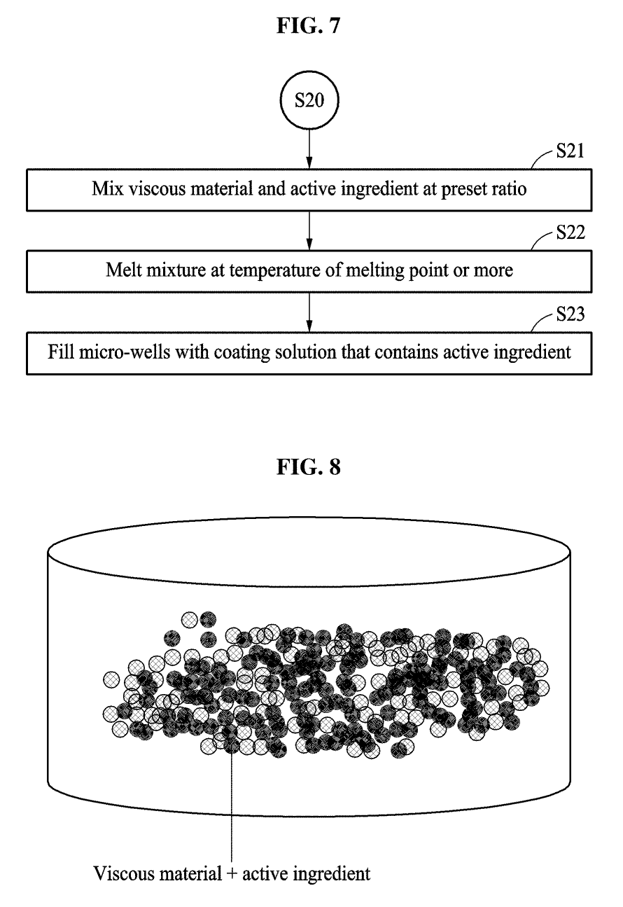

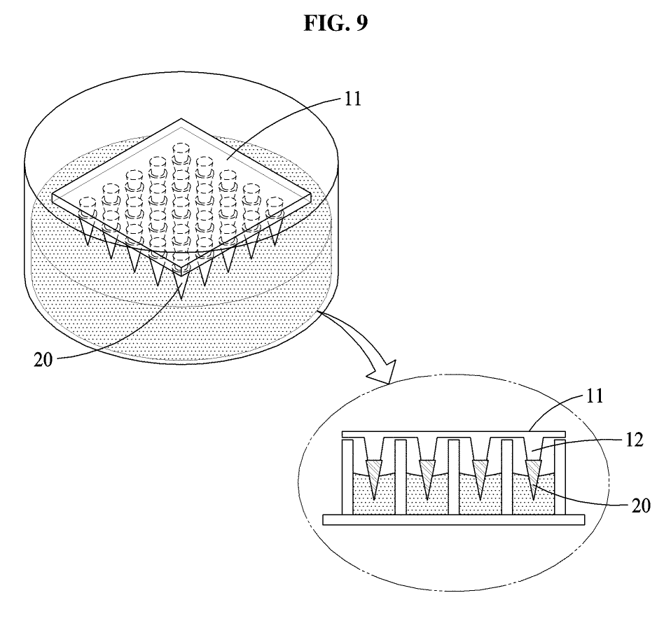

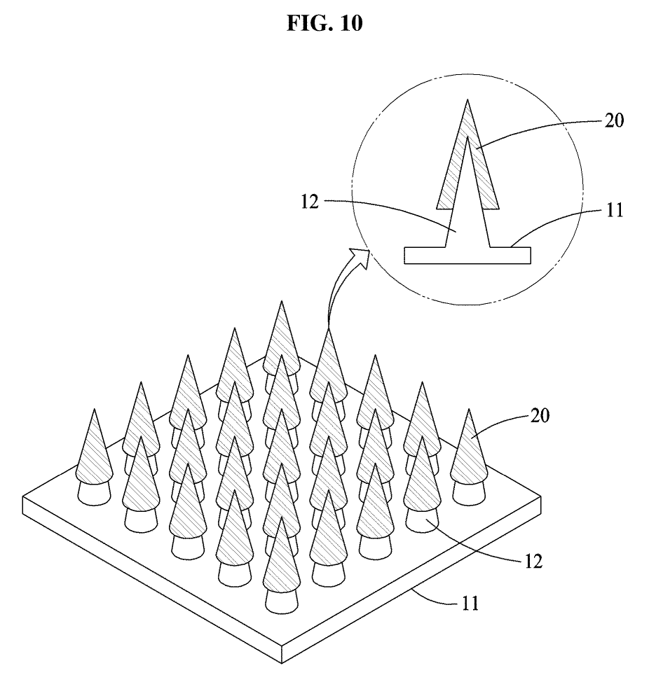

FIG. 7 is a flowchart illustrating an operation of preparing a coating solution containing an active ingredient of a microneedle according to example embodiments, FIG. 8 illustrates an example of mixing a viscous material having a melting point greater than or equal to a room temperature and an active ingredient at a preset ratio and melting the mixture at a temperature of the melting point or more to prepare a coating solution of an active ingredient coating portion of a microneedle according to example embodiments, FIG. 9 illustrates a state in which a needle body portion of a microneedle is dipped into a coating solution containing an active ingredient according to example embodiments, and FIG. 10 illustrates an example of drying a microneedle coated with an active ingredient coating portion at a room temperature according to example embodiments.

Referring to FIGS. 7 through 10, operation S20 of preparing a coating solution of the active ingredient coating portion 20 used to coat the needle body portion 12 may include operation S21 of mixing a viscous material having a melting point and an active ingredient at a preset ratio, operation S22 of melting the mixture of the viscous material and the active ingredient at a temperature of, for example, 120° C. in the case of maltose, the melting point or more, and operation S23 of filling micro-wells with the coating solution that contains the active ingredient. For example, the coating solution may be prepared by mixing the viscous material, the active ingredient, and water-soluble additives in the deionized water or a PBS solution at a preset ratio. The prepared coating solution may fill in the micro-wells and thereby dip-coat the needle body portion 12.

The viscous material having the melting point and to be mixed with the active ingredient during a process of preparing the coating solution of the active ingredient coating portion 20 may include at least one of maltose, lactose, trehalose, cellobiose, isomaltose, turanose, and lactulose. The melting point of the viscous material included in the the active ingredient coating portion 20 may be greater than or equal to a room temperature, for example, 25° C.

The coating solution of the active ingredient coating portion 20 may be prepared by melting at least one of the water-soluble viscous materials each having a melting point and the active ingredient at a preset ratio. Desirably, in the case of the active ingredient coating portion 20, a weight % ratio of viscous material:active ingredient may be within the range of 9:1 to 1:9, desirably, within the range of 1:1 to 1:2.

Here, the active ingredient may include at least one of α-interferon, β-interferon for multiple sclerosis (MS), erythropoietin, follitropin β, follitropin α, G-CSF, GM-CSF, human chorionic gonadotropin, luteinizing hormone, salmon calcitonin, glucagon, GNRH antagonist, insulin, human growth hormone, filgrastim, heparin, low-molecular-weight heparin, somatropin, Japanense encephalitis vaccine, rotavirus vaccine, Alzheimer vaccine, artery hardening vaccine, cancer vaccine, nicotine vaccine, diphtheria vaccine, tetanus vaccine, pertussis vaccine, lyme disease vaccine, rabies vaccine, dipolcoccus pneumoniae vaccine, yellow fever vaccine, cholera vaccine, vaccinations-exanthem vaccine, tuberculosis (TB) vaccine, German measles vaccine, measles vaccine, epidemic parotitis vaccine, botulinus vaccine, herpesvirus vaccine, other DNA vaccine, hepatitis (type) B vaccine, hyaluronic acid, coenzymeq10, chitosan, botox, vitamin and vitamin derivatives, hydroxy acid, tetracycline, oxytetracycline, doxycycline, minocycline, benzocaine, mepivacaine, lidocaine, prilocaine, bupivacaine, etidocaine, articaine, procaine, propoxycaine, tetracaine, ropivacaine, butacaine, piperocaine, cocaine, chloroprocaine, proparacaine, and dyclonine.

Once coating is completed by dipping the needle body portion 12 manufactured through molding into the prepared coating solution that contains the active ingredient, the needle body portion 12 coated with the coating solution may be dried at a room temperature in operation S40.

Since the needle body portion 12 is coated with the coating solution prepared by mixing the viscous material, such as maltose and the like, having a melting point and the active ingredient and by melting the mixture at a temperature of the melting point of the viscous material or more, and is dried at a room temperature, there is no need to wait until the moisture of the coating solution evaporates. Accordingly, it is possible to dry the needle body portion 12 relatively quickly.

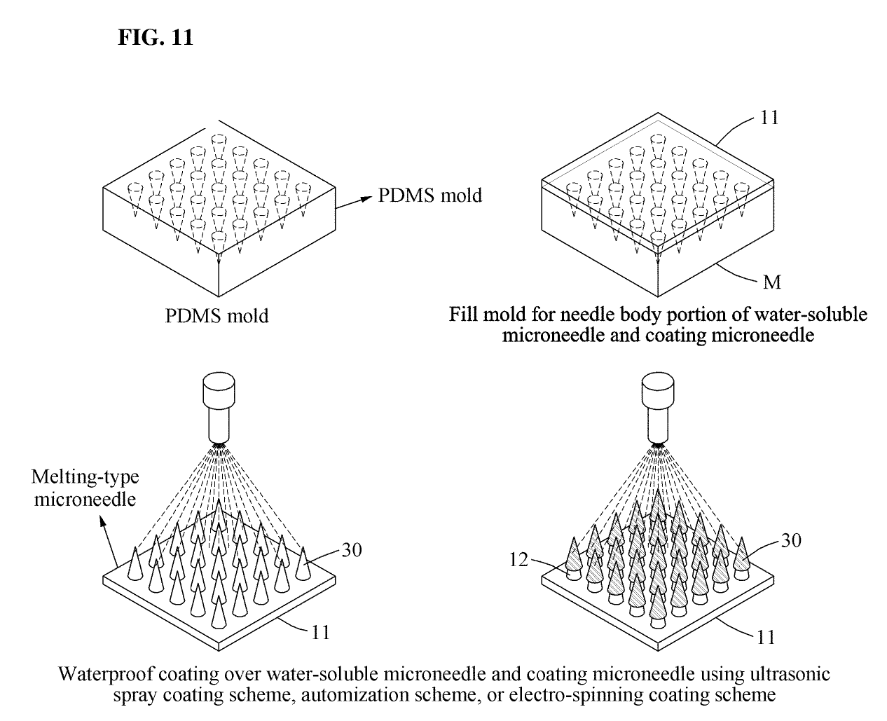

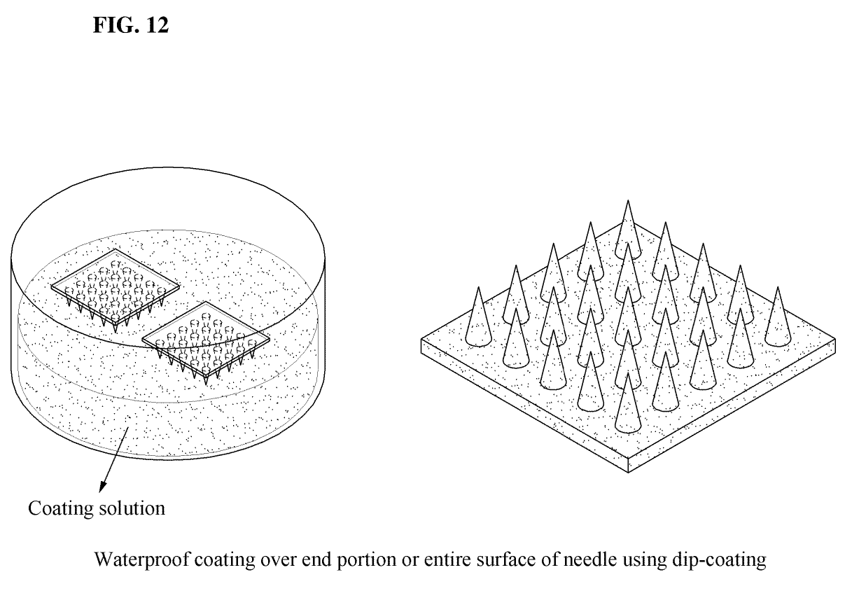

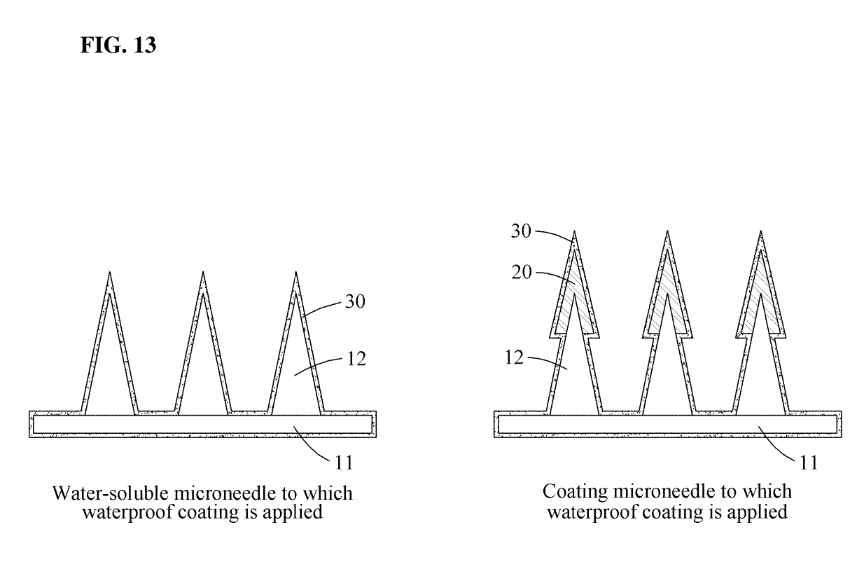

FIG. 11 illustrates an example of a process of coating a dried microneedle with a lipid-based material or a mineral-based material using an ultrasonic spray coating scheme or an electro-spinning coating scheme according to example embodiments, FIG. 12 illustrates an example of dipping a dried microneedle into a lipid-based material or a mineral-based material according to example embodiments, and FIG. 13 illustrates an example of drying a microneedle coated with a lipid-based material or a mineral-based material at a room temperature according to example embodiments.

Referring to FIGS. 11 through 13, in operation S50, the needle body portion 12 coated with the active ingredient coating portion 20 and dried at a room temperature may be coated with the waterproof coating portion 30. The waterproof coating portion 30 may be formed using a lipid-based material or a mineral-based material to prevent deformation of the needle body portion 12 and the active ingredient coating portion 20 formed using a water-soluble material.

The lipid-based or mineral-based waterproof coating portion 30 may include at least one of beeswax, oleic acid, soy fatty acid, castor oil, phosphatidylcholine, d-α-tocopherol/vitamin E, corn oil mono-di-tridiglycerides, castor oil, corn oil, cottonseed oil, olive oil, peanut oil, peppermint oil, safflower oil, sesame oil, soybean oil, hydrogenated vegetable oil, hydrogenated soybean oil, caprylic/capric triglycerides derived from coconut oil or palm see oil, phosphatidylcholine, polyethylene (PE), polypropylene (PP), polytetrafluoroethylene (PTFE), polymethylmethacrylate (PMMA), ethylene vinyl acetate (EVA), polycaprolactone (PCL), polyurethane (PU), polyethylene terephthalate (PET), polyethylene glycol (PEG), polyvinyl alcohol (PVA), poly lactide (PLA), poly lactic-co-glycolic acid (PLGA) polymer, polyglycolide (PGA), wax (paraffin cholesterol), glycerin, chitin, lecithin, animal beef tallow, vegetable stearin, low-grade saturated fatty acid, monosaturated fatty acid, tristearins, fatty acid mineral salt (zinc, calcium, magnesium stearate), and fatty acid zinc salt (stearic acid, palmitic acid, lauric acid), or includes the mixture thereof.

In operation S50 of coating the needle body portion 12 coated with the active ingredient coating portion 20 with the waterproof coating portion 30, the lipid-based material or the mineral-based material may be used to coat an upper end or the entire outer periphery of the needle body portion 12 dried at the room temperature in a state in which the needle body portion 12 is coated with the active ingredient coating portion 20. For example, the waterproof coating portion 30 may be formed by melting biodegradable fat or hydrophobic material in ethanol.

The needle body portion 12 coated with the active ingredient coating portion 20 may be coated using an ultrasonic spray coating scheme for spraying the lipid-based material or the mineral-based material. In this case, a spray device may be installed in an upper portion of the needle body portion 12 coated with the active ingredient coating portion 20. A portion of or the entire surface of the needle body portion 12 coated with the active ingredient coating portion 20 may be coated by spraying the lipid-based coating solution from the spray device.

Also, the needle body portion 12 coated with the active ingredient coating portion 20 may be coated by being dipped into the lipid-based coating solution.

In detail, the waterproof coating portion 30 used to coat the needle body portion 12 coated with the active ingredient coating portion 20 and thereby dried may be prepared by mixing, into an ethanol solution, beeswax of 3 mg/ml, cholesterol of 32.5 mg/ml, phosphatidylcholine of 550 mg/ml, palmitic acid of 210 mg/ml, lauric acid of 450 mg/ml, and stearic acid of 70 mg/ml. The prepared waterproof coating portion 30 enables even coating.

In operation S60, the microneedle 1 coated with the waterproof coating portion 30, for example, the lipid-based material, may be dried at a room temperature.

Since the needle body portion 12 coated with the active ingredient coating portion 20 is coated with the lipid-based material, an upper end portion of the microneedle 1 may be maintained to be in a sharp shape even in a moisture environment.

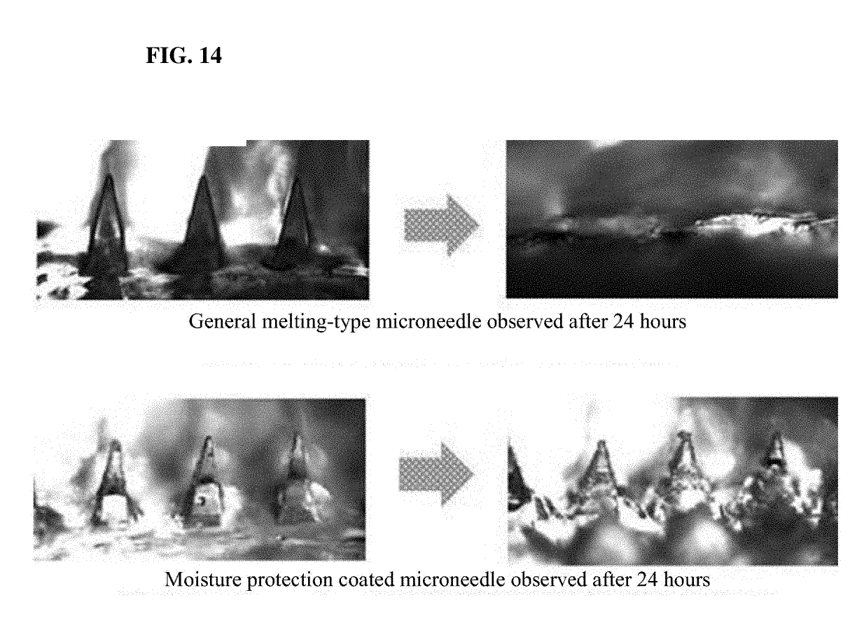

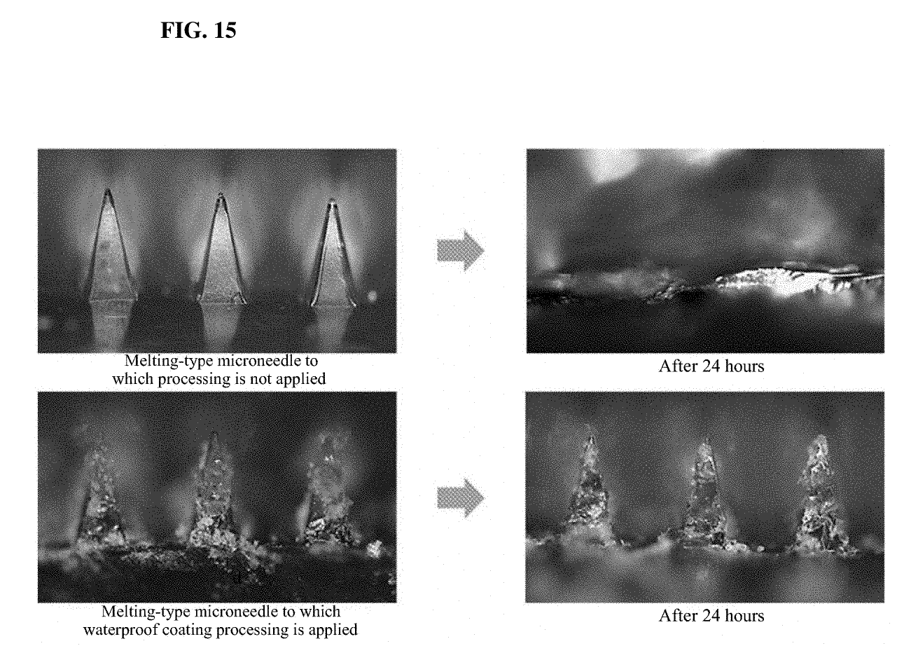

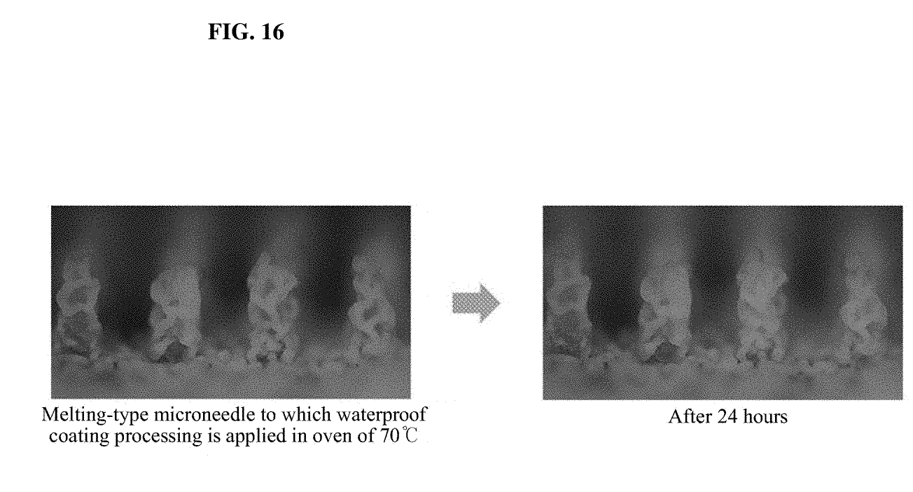

FIGS. 14 and 15 illustrate an example of comparing a shape of a general melting-type microneedle and a shape of a microneedle coated with a moisture protection solution in a moist environment after 24 hours, and FIG. 16 illustrates an example of a microneedle observed after 24 hours by putting a moisture-sensitive microneedle in an oven of 70° C. and performing waterproof coating.

Referring to FIGS. 14 through 16, the outer periphery of the needle body portion 12 is coated with the active ingredient coating portion 20 that includes the active ingredient and the viscous material having a relatively high solubility rate. Thus, when the needle body portion 12 is inserted into the biological skin tissue, a fixed amount of active ingredients may be quickly delivered to the inside of the biological tissue.

To protect the microneedle 1 or water-sensitive materials in a moisture environment, the surface of the microneedle 1 is to be uniformly coated. For example, the needle body portion 12 coated with the active ingredient coating portion 20 may be dip-coated using 150 mg/ml of palmitic acid solution used for the waterproof coating portion 30. In detail, the palmitic acid solution corresponding to an amount at which the microneedle 1 can be sufficiently dipped into a petri dish may be prepared and the water-sensitive microneedle 1 may be dipped for about five seconds and then taken out and be dried at a room temperature.

As another example, 150 mg/ml of palmitic acid solution may be placed in the petri dish and the water-sensitive microneedle 1 may be dipped into the palmitic acid solution for about five seconds and then taken out and placed in an oven of 70° C. Through this, the palmitic acid solution may be uniformly distributed over the overall surface of the microneedle 1 by evaporating ethanol instantaneously (see FIG. 16).

Further, coating may be performed by uniformly spraying 150 mg/ml of palmitic acid solution using an electro-spinning machine or an ultrasonic coating machine. The coated microneedle 1 may be dried at a room temperature.

Accordingly, compared to a melting-type microneedle to which processing is not applied, a melting-type microneedle to which waterproof coating processing is applied by coating the microneedle with the palmitic acid solution configured as above may maintain a needle shape although the same period of time is elapsed.

Although a few example embodiments have been shown and described, the present disclosure is not limited to the described embodiments. Instead, it would be appreciated by those skilled in the art that changes may be made to these embodiments without departing from the principles and spirit of the disclosure, the scope of which is defined by the claims and their equivalents.

What is claimed is:

1. A microneedle comprising:
a needle body portion formed using a water-soluble material;
an active ingredient coating portion configured to coat the surface of the needle body portion, and including an active ingredient transferred to the biological tissue; and
a waterproof coating portion configured to coat at least a portion of the surface of the active ingredient coating portion, and to protect the needle body portion and the active ingredient coating portion from moisture.

2. The microneedle of claim 1, wherein the needle body portion includes at least one of a viscous material and the active ingredient transferred to the biological tissue.

3. The microneedle of claim 2, wherein the viscous material included in the needle body portion includes at least one of carboxymethyl cellulose (CMC), hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polylactic acid (PLA), polyglycolide (PGA), poly lactic-co-glycolic acid (PLGA), pullulan polyanhydride, polyorthoester, polyetherester, polycarprolactone, polyesteramide, poly (butyric acid), poly (valeric acid), polyurethane, polyacrylate, ethylene-vinyl acetate polymer, acrylic substituted cellulose acetate, non-degradable polyurethane, polystyrene, polyvinyl chloride, polyvinylidene fluoride, poly (vinyl imidazole), chlorosulphonate polyolefins, polyethylene oxide, poly vinyl pyrrolidone (PVP), polyethylene glycol (PEG), polymethacrylate, hydroxypropyl methyl cellulose (HPMC), ethylcellulose (EC), hydroxypropyl cellulose (HPC), carboxymethyl cellulose, cyclodextrin, copolymer of monomers constituting the polymer, and cellulose.

4. The microneedle of claim 1, wherein the needle body portion is formed using biodegradable polymer resin.

5. The microneedle of claim 1, wherein the active ingredient coating portion includes a viscous material having a melting point greater than or equal to a room temperature and the active ingredient transferred to the biological tissue.

6. The microneedle of claim 5, wherein the viscous material included in the active ingredient coating portion includes at least one of maltose, lactose, trehalose, cellobiose, isomaltose, turanose, and lactulose.

7. The microneedle of claim 6, wherein the viscous material is included in the needle body portion, and
a solubility of the viscous material included in the active ingredient coating portion against the moisture is greater than a solubility of the viscous material included in the needle body portion against the moisture.

8. The microneedle of claim 2, wherein the active ingredient included in the needle body portion and the active ingredient coating portion includes at least one of α-interferon, β-interferon for multiple sclerosis (MS), erythropoietin, follitropin β, follitropin α, G-CSF, GM-CSF, human chorionic gonadotropin, luteinizing hormone, salmon calcitonin, glucagon, GNRH antagonist, insulin, human growth hormone, filgrastim, heparin, low-molecular-weight heparin, somatropin, Japanese encephalitis vaccine, rotavirus vaccine, Alzheimer vaccine, artery hardening vaccine, cancer vaccine, nicotine vaccine, diphtheria vaccine, tetanus vaccine, pertussis vaccine, lyme disease vaccine, rabies vaccine, dipolcoccus pneumoniae vaccine, yellow fever vaccine, cholera vaccine, vaccinations-exanthem vaccine, tuberculosis (TB) vaccine, German measles vaccine, measles vaccine, epidemic parotitis vaccine, botulinus vaccine, herpesvirus vaccine, other DNA vaccine, hepatitis (type) B vaccine, hyaluronic acid, coenzymeq10, chitosan, botox, vitamin and vitamin derivatives, hydroxy acid, tetracycline, oxytetracycline, doxycycline, minocycline, benzocaine, mepivacaine, lidocaine, prilocaine, bupivacaine, etidocaine, articaine, procaine, propoxycaine, tetracaine, ropivacaine, butacaine, piperocaine, cocaine, chloroprocaine, proparacaine, and dyclonine.

9. The microneedle of claim 1, wherein the waterproof coating portion includes a lipid-based material or a mineral-based material.

10. The microneedle of claim 9, wherein the lipid-based material or the mineral-based material included in the waterproof coating portion includes at least one of beeswax, oleic acid, soy fatty acid, castor oil, phosphatidylcholine, d-α-tocopherol/vitamin E, corn oil mono-di-tridiglycerides, castor oil, corn oil, cottonseed oil, olive oil, peanut oil, peppermint oil, safflower oil, sesame oil, soybean oil, hydrogenated vegetable oil, hydrogenated soybean oil, caprylic/capric triglycerides derived from coconut oil or palm see oil, phosphatidylcholine, polyethylene (PE), polypropylene (PP), polytetrafluoroethylene (PTFE), polymethylmethacrylate (PMMA), ethylene vinyl acetate (EVA), polycaprolactone (PCL), polyurethane (PU), polyethylene terephthalate (PET), polyethylene glycol (PEG), polyvinyl alcohol (PVA), poly lactide (PLA), poly lactic-co-glycolic acid (PLGA) polymer, polyglycolide (PGA), wax (paraffin cholesterol), glycerin, chitin, lecithin, animal beef tallow, vegetable stearin, low-grade saturated fatty acid, monosaturated fatty acid, tristearins, fatty acid mineral salt (zinc, calcium, magnesium stearate), and fatty acid zinc salt (stearic acid, palmitic acid, lauric acid), or includes the mixture thereof.

11. A method of manufacturing a microneedle, the method comprising:
manufacturing a needle body portion of the microneedle to be in a preset shape;
preparing a coating solution in which a viscous material having a melting point greater than or equal to a room temperature and an active ingredient transferred to the biological tissue are mixed;
coating the needle body portion of the micro needle with the coating solution;
forming an active ingredient coating portion on the surface of the needle body portion of the microneedle by drying the needle body portion of the microneedle coated with the coating solution at the room temperature;
coating at least a portion of the surface of the active ingredient coating portion with a waterproof coating portion; and
drying the waterproof coating portion at the room temperature.

12. The method of claim 11, wherein the manufacturing of the needle body portion of the microneedle comprises:
preparing a mold for molding the needle body portion of the microneedle;
molding the needle body portion of the microneedle; and
separating the needle body portion of the microneedle from the mold.

13. The method of claim 12, wherein the molding of the needle body portion of the microneedle comprises:
manufacturing the needle body portion of the microneedle by filling the mold with the viscous material and the active material transferred to the biological tissue and drying the same, or by applying a centrifugal process.

14. The method of claim 12, wherein the molding of the needle body portion of the microneedle comprises:
filling the mold with the viscous material and the active ingredient transferred to the biological tissue and manufacturing the needle body portion of the microneedle using a vacuum pump.

15. The method of claim 12, wherein the molding of the needle body portion of the microneedle comprises:
heating biodegradable polymer resin at a preset temperature in a vacuum oven.

16. The method of claim 12, wherein the mold is a structure that includes at least one of polydimethylsiloxane (PDMS), a type of polymer used for the mold, polyurethane, metal, an aluminum biocompatible material, water-soluble polymer, fat-soluble polymer, and amphiphilic polymer, and
the fat-soluble polymer and the amphiphilic polymer include at least one of hydroxypropyl cellulose (HPC), hydroxypropyl methyl cellulose (HPMC), polycaprolactone (PCL), polyglycolide (PGA), polylactic acid (PLA), poly lactic-co-glycolic acid (PLGA) polymer, poly vinyl pyrrolidone (PVP), polyethylene glycol (PEG), poly ethylene oxide (PEO), poly propylene oxide (PPO), poly vinyl methyl ether (PVME), PMA (poly (methyl) acrylate)s, propylene glycol, poly (ester amide), poly (butyric acid), acrylamide (acrylic amide), acrylic acid, hyaluronic acid (HA), and gelatin.

17. The method of claim 11, wherein the preparing of the coating solution comprises:
mixing the viscous material and the active ingredient at a preset ratio; and
melting the mixture of the viscous material and the active ingredient at a temperature greater than or equal to the melting point of the viscous material.

18. The method of claim 11, wherein the coating of the needle body portion of the microneedle with the coating solution comprises dip-coating or entirely coating the needle body portion of the microneedle with the coating solution.

19. The method of claim 18, wherein the coating solution is a lipid-based or mineral-based coating solution.

20. The method of claim 11, wherein the drying of the waterproof coating portion at the room temperature comprises coating the surface of the active ingredient coating portion with a lipid-based material or a mineral-based material using an ultrasonic spray coating scheme, an automization coating scheme, or an electro-spinning coating scheme, or dip-coating the needle body portion of the microneedle with a lipid-based or mineral-based coating solution.
